(19) United States
(12) Patent Application Publication
(10) Pub. No.: US 2004/0064344 A1
(43) Pub. Date: Apr. 1, 2004

(54) Method for obtaining dynamic informed consent

(76) Inventors: Ronald J. Link, Tampa, FL (US); Arthur Epstein, Great Neck, NY (US); Kenneth Keith, Rockville Center, NY (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/607,526

(22) Filed: Jun. 27, 2003

Related U.S. Application Data
(63) Continuation-in-part of application No. 10/299,811, filed on Nov. 20, 2002.
(60) Provisional application No. 60/332,223, filed on Nov. 20, 2001.

(51) Int. Cl. G06F 17/60; A61B 5/00

(52) U.S. Cl. 705/2; 600/300

(57) ABSTRACT

A method for providing a dynamic, customized patient consent form for informing a patient of the risk of undergoing a surgery or therapeutic treatment by inputting dynamic and semi-static data to create a rule-based algorithm for calculating different risks of undergoing the surgery; drafting different explanatory paragraphs of the different risks; inputting individual patient data; creating a customized risk assessment and assembling a customized consent form by selecting the appropriate explanatory paragraphs.

### CROSS REFERENCE TO RELATED APPLICATION

This application is a continuation-in-part of U.S. application Ser. No. 10/299,811, filed Nov. 20, 2002, and also claims priority from U.S. Provisional Application No. 60/332,223, filed Nov. 20, 2001.

### FIELD OF THE INVENTION

The invention relates to determining a patient's risks in undergoing surgery or other therapeutic treatment, and more particularly to creating a dynamic informed consent form that displays to a patient a customized risk assessment for such surgery or treatment.

### SUMMARY OF THE INVENTION

The invention pro vides an individualized consent form that alerts a patient as much as measurably possible to the probability of the patient's having a successful outcome in undergoing a surgical procedure or therapeutic regimen while simultaneously advising the health care provider of relative risks of procedures or therapies. The invention also provides a computer-implemented method for creating the consent form, which may be created through an internet guided-entry user interface or through a computer terminal or workstation that may be connected to a local/wide area network.

For convenience of reference in the remainder of the description of the invention, the text will often refer to “surgery” as the patient treatment under consideration. However, it shall be understood that the process of the present invention is equally adapted to other therapeutic processes and procedures (by way of example: hemodialysis, chemotherapy, radiological treatment, laser eye surgical procedures, and others).

Using data describing contraindications and complications from FDA guidelines, public manufacturer data, clinical results from professional industry conferences, interdisciplinary scientific literature and negative case law, as well as dynamic data about surgeons' outcome and results history for the surgery, the method calculates in real time a customized risk assessment in terms of the probability of a successful outcome for the patient undergoing a surgery or treatment by a certain physician using a selected devices or therapy. Based on the customized risk analysis, the method causes a consent form to be generated in real time, which comprises standardized and individualized paragraphs explaining the risks associated with the surgery or treatment for the patient.

The invention also comprises a method whereby a real-time, dynamic informed consent form is created at a computer work station in a contracted health care provider's office by processing specific complication rates and outcome measures for a surgical device(s) or therapies with which the contracted provider performs surgery or treatment. Clinical basis process data is fed through algorithms of a rule-based informed consent engine that mathematically arrive at a level of definite and/or probable range or risk for each individual patient. An online word-processing program displays the real-time dynamic informed consent form, which includes outcomes analysis, clinical (pre-and post-op measurements) patient satisfaction (subjective reporting and graphic interface generation), graphics program presenting illustrative charts and graphs, identification of contraindications and complications for a specific surgery and weighted risk based on individual patient pre-op profile. The word processing program changes the paragraphs in the real-time dynamic informed consent form to display the individual patient degree of risk when patient degree of risk is formulated. The patient may be shown through the computer photographs or other graphical illustrations of different kinds of outcomes such as visual aberrations possibly resulting from the surgery or treatment, and patient testimonials, both positive and negative, to augment written advisement. A printout may be provided for the patient to take home and review.

### BRIEF DESCRIPTION OF THE DRAWINGS

FIG. 1 illustrates a prior art method of formulating informed consent forms.

FIG. 2 illustrates one embodiment of the present method of developing a dynamic informed consent form

FIG. 3 illustrates further detail of the embodiment of the process shown in FIG. 2.

### DETAILED DESCRIPTION

The following definitions are used in this specification:

Contraindications comprise measurable interdisciplinary variables that have pre- and post-operation (or therapy) significance (either absolutely or relatively). Three categories of contraindication are Established, Emerging and Unknown. Contraindications are obtained by the dynamic informed consent method of the present invention from both Semi-Static Data and Dynamic Data.

Semi-static Data comprise information about established or emerging contraindications, which are assigned a mathematical value when possible based on their identification as either established or emerging. Such data are gleaned from the following sources, which list is not intended to be exhaustive:

FDA guidelines, as published;

Individual manufacturer data, when publicly accessible;

Professional industry conferences in terms of clinical results presented to peers in the medical profession;

Interdisciplinary scientific literature, including medical journals and individual clinic and/or physician studies;

Consumer groups, including scientific research done by consumer advocacy groups on complications sustained from surgery and other therapies.

Dynamic Data comprise outcome and results history data of surgeons and practitioners performing a particular form of surgery or therapeutic treatments. These data do not include individual surgeon or patient identifiers.

Complications comprise identifiable and measurable compromises of physical condition and/or function, which may be permanent or which diminish or disappear over time. Common examples of these are post-operative infections (physical condition), and neurological motor impairment (physical function). Three categories of complications are: Established, Emerging and Unknown.

The present method provides a comprehensive event-driven consent form which alerts a patient as much as measurably possible to his or her individual degree of risk in developing possible complications in undergoing a surgery or other therapeutic treatment. The method goes beyond the present strategy for using chronological-hierarchical information to determine a patient's surgical risk by measuring variables and using streaming data, which are processed by algorithms of an informed consent engine that mathematically arrive at a level of definite and/or probable range or risk for each individual patient.

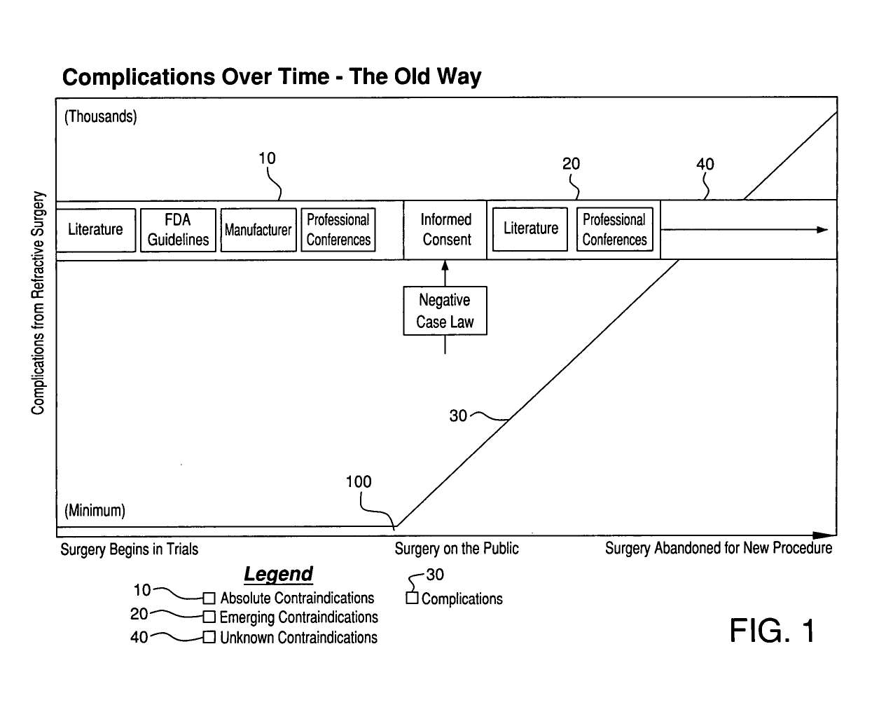

FIG. 1 illustrates a prior-art method for providing a patient an informed consent form. As shown, the prior process includes information on the absolute contraindications 10 of refractive laser surgery for correcting vision, which is gathered from the medical, literature, FDA guidelines, the manufacturer's warnings and professional conferences. These absolute contraindications have traditionally been used to formulate informed consent forms and to assess risks for patients considering the procedure at the point 100 when the, surgery commences on the general public.

As FIG. 1 shows, once the “informed” consent is formulated, there is little or no opportunity in the prior art process to re-formulate the informed consent so as to include the mounting evidence 30 of complications resulting from the procedure. That is, the basis for the informed consent form in the prior art method constitutes almost entirely the absolute contraindications originally formulated before actual practice of the surgical technique on the public.

To the point, even though the medical literature and reports in professional conferences may apprise medical personnel of emerging contraindications 20 of the procedure, the patients may not be receiving this subsequent information. Moreover, the patient often is not informed of the mounting data 30 of complications occurring in and or related to the surgery. And, to the extent that there are unknown contraindications 40 to the surgical procedure, the patient cannot know of these either.

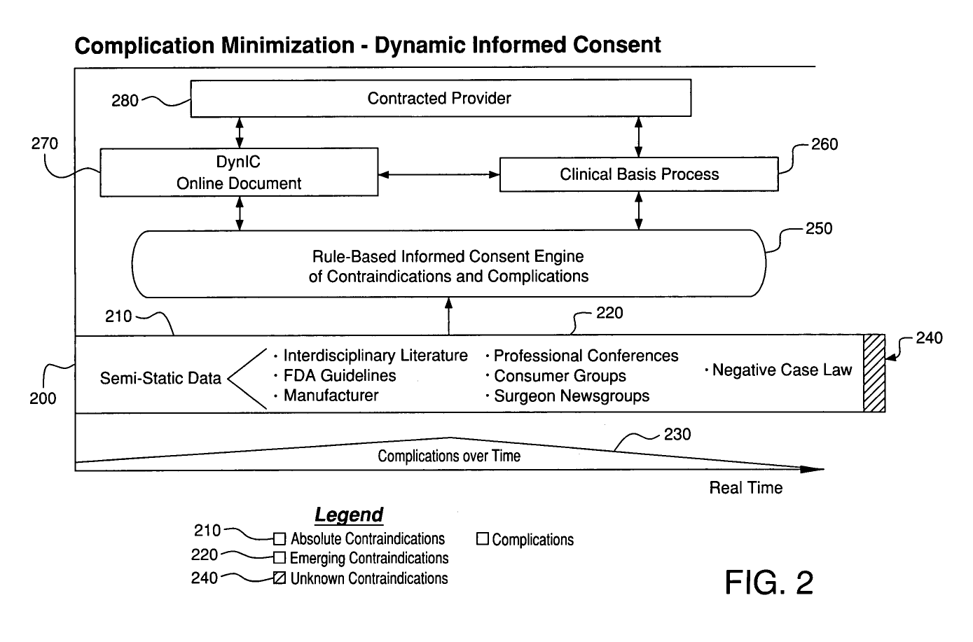

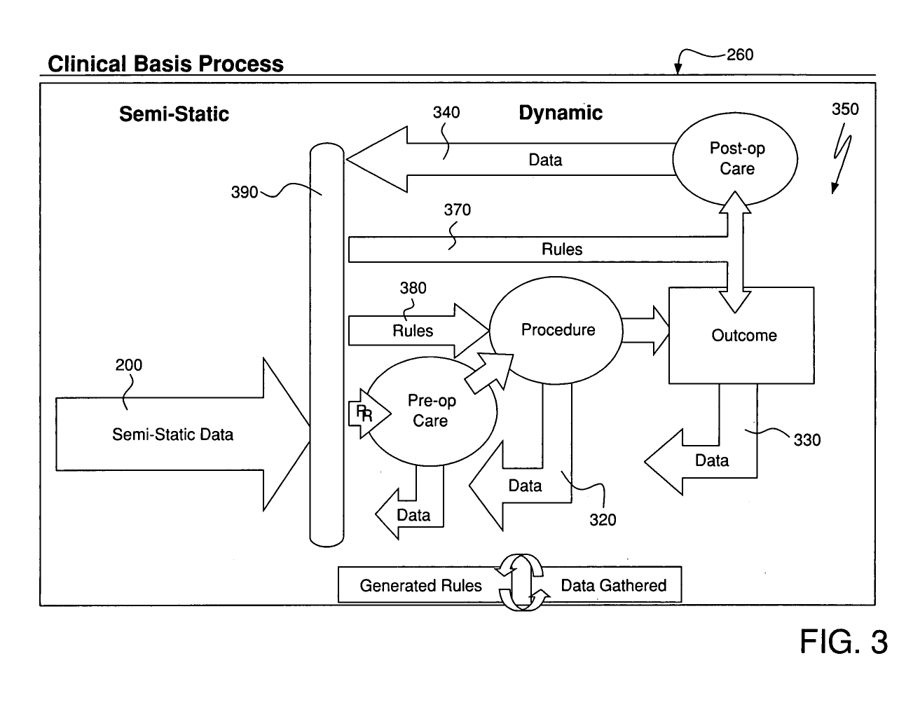

FIG. 2 illustrate's an exemplary embodiment of the present method in which the data is continually accumulated, processed, and presented to the patient to provide an up to the moment truly informed consent. A health care provider, such as a hospital, a clinic, physician's practice group or a sole practitioner contracts to become a participating member. Participation in the present process provides members and their patients the ability to calculate and print out an individualized risk assessment for undergoing surgery. Semi-static data 200, which comprises information on absolute contraindications 210 and emerging contraindications 220, emerging data relating to complications 230 occurring as a result of the surgery are input into the rule-based, informed consent engine 250. The method then calculates, using a clinical basis process 260 (shown in more detail in FIG. 3), a customized risk analysis for the patient contemplating a procedure.

Through a word processing function, the method prints out a customized Dynamic Informed Consent Form 270 which includes specific paragraphs relating to the patient's customized risk analysis. By contracting to acquire the present process of creating dynamic informed consent forms, a contracted provider can offer a customized informed consent form that details the risks of surgery for its patients contemplating the procedure.

FIG. 3 illustrates an embodiment of the clinical basis process 300 in which Semi-static data 200 and Dynamic data 350 are used to generate the rule-based algorithm 390, which shapes the Dynamic Informed Consent Form. The 390, formulates rules of risks relating to the surgical procedure 380 and rules of risks developed from analyzing data on post-operative events and outcomes 370. In turn, the generated rules 370 and 380 do not remain static but are re-evaluated and re-generated upon the input of data 310 on patients' pre-operative and post-operative care 340, data 320 on the surgical procedure and data 330 on the positive and negative outcomes of the surgery as performed by surgeons associated with the health care provider.

The clinical basis process 300 is a real time, iterative calculation of the risks for an individual patient, considering not only the semi-static data of contraindications but also the dynamic data on complications and emerging contraindications, which relate to the outcome and result track record of particular surgeons associated with a contracted health care provider. In other words, the clinical basis process 300 evaluates an individualized patient risk assessment from all the dynamic data on patient outcomes and results of surgeries performed by various surgeons of a contracted provider as well as from the semi-static data relating to surgical devices used for the procedure. The same process is equally adapted to the evaluation and risk assessment related to other medical therapeutic processes and treatments and those who provide them.

Once calculated, the risk assessment is presented in the form of a customized dynamic informed consent form. This is accomplished by drafting separate sentences or paragraphs that explain different risks and creating a calculus that associates different explanatory sentences or paragraphs with different patient conditions and for different surgeons.

The method is iterative and dynamic in that the clinical basis process 300 may be continually updated with real time data to provide a continually updated rule-based algorithm. That is, as updated data on surgeons' and patients' outcomes and results history are acquired, the rule-based algorithm both fine-tunes the surgical risks associated, with various patient conditions and different, surgeons and updates the calculus that associates the risk explanations with these conditions and surgeons. By updating the risk assessment algorithm and the calculus for associating explanations with surgery conditions particularly by inputting dynamic data, the resultant informed consent form may be continually updated and customized for individual patients.

Thus, the process of the present invention is a method of data gathering, processing, and presentation that provides a patient with a real-time informed consent to medical procedures. The process includes a mechanism for data mining and storage of existing relevant information, including, though not limited to: FDA/governmental approval data, scientific and journal reports, validated anecdotal information, and previously gathered dynamic data.

An algorithm-based data engine dynamically processes and analyzes static and dynamic data on a continuing, recursive basis. The result is a presentation in real-time that identifies risk in surgical and medical procedures that provides a patient with a live, state of the moment informed consent based on existing indications and warnings, literature, and iteratively processed data gathered from other patients. The process can also generate warnings based on all available data when such warnings would appropriate.

The process helps guide the patient by more fully exposing risk and the surgeon (or other practitioner) by identifying risk factors and relating patient-specific characteristics to a treatment outcome. The data may also be used to automate and improve post-approval surveillance as likely required by any government or private organization. It may also assist inequality assurance for physicians, surgeons, hospitals, device manufacturers and surgical and treatment centers.

Dynamic information is gathered preoperatively standardized to exclude sources of bias or site variation; perioperatively using self-validating methodology, and postoperatively during examinations and through patient surveys consented during the initial data gathering period.

According to the invention, gathered data is applied recursively to generate an individualized, patient specific, informed consent for medical and surgical procedures that will unify all generally known risks, criteria, contraindications and alternatives to specific surgical procedures or medical treatments. The informed consent is based upon patient-provided information, devices assessment (where applicable), and pre-operative/treatment testing assessed against the body of existing medical research and a database of previous outcomes from treatments performed after standardized pre-treatment assessment. The gathered data is used to provide a relative risk assessment for both patient and doctor gathered information that can be used for post-approval FDA surveillance or any government or private organizations or institutions. The invention is adaptable to (but not limited to) internet-based data gathering methodology which will help assure standardization. The invention employs recursive application of customized algorithms to gathered data to help refine risk endpoints and provide clinical guidance.

Certain modifications and improvements will occur to those skilled in the art upon a reading of the foregoing description. It will be readily apparent that such modifications and improvements could be made therein without departing from the scope of the invention as defined in the following claims.

What is claimed is:

1. A computer-implemented process for informing a patient of the risk of undergoing a treatment, said method comprising the steps of:
a) gathering semi-static data relating to contraindications to and complications associated with the treatment;
b) gathering dynamic data relating to experienced contraindications to and complications associated with the treatment, said dynamic data comprising information about the treatment conduct, its result, and patient response over time;
c) from the gathered semi-static data and dynamic data, creating a rule-based algorithm for calculating the risks of undergoing the treatment,
f) acquiring relevant data of an individual patient;
g) calculating a customized personal risk assessment for the individual patient;
h) presenting the customized personal risk assessment to the patient.

2. The process of claim 1, wherein the step of creating a rule-based algorithm for calculating the risks of undergoing the treatment comprises:
periodically updating both the semi-static data and dynamic data.

3. The process of claim 1, wherein the step of creating a rule-based algorithm for calculating the risks of undergoing the treatment comprises:
recursively processing the rules governing the risk assessment relating to any treatment based on periodic updates to one or both of the semi-static and dynamic data.

4. The process of claim 1, further comprising the step:
formulating text material in the form of sentences that list risk factors and outcome information for display to the patient based on information gathered in the process.

5. The process of claim 4, further comprising the step:
creating a calculus that associates text sentences with the risk information to be presented to the patient.

6. The process of claim 1, wherein the step of gathering dynamic data relating to experienced contraindications to and complications associated with the treatment includes:
identifying a particular treatment provider, and
incorporating data relating to the treatment provider.

7. The process of claim 6, wherein the step of incorporating data relating to the treatment provider further comprises:
gathering and including data on'the treatment provider's outcome history associated with the treatment.

8. The process of claim 7, wherein the step of incorporating data relating to the treatment provider further comprises:
gathering and including data on the treatment provider's complication history in providing the subject treatment.

9. The process of claim 1, wherein the step of gathering dynamic data relating to experienced contraindications to and complications associated with the treatment includes:
gathering information about the pre-operative and post-operative care of the treatment provider's patients.

10. A computer-implemented process for informing a patient of the risk of undergoing a treatment, said method comprising the steps of:
a) gathering semi-static data relating to contraindications to and complications associated with the treatment;
b) acquiring relevant data of an individual patients;
c) calculating a customized personal risk assessment for the individual patient;
d) presenting to the patient an individualized risk assessment based on the gathered data.

11. The process of claim 10, further comprising the step:
printing the individualized risk assessment as an informed consent form.

12. The process of claim 10, further comprising the step:
gathering performance data relating to a particular treatment provider, including information on treatment outcomes for patients treated by that provider.

13. The process of claim 12 further comprising the step:
from the gathered semi-static data, the patient data, and treatment provider data, creating a rule-based algorithm for calculating the risks of undergoing the treatment.

14. The process of claim 13 further comprising the step:
recursively processing the rules governing the risk assessment relating to any treatment based on periodic updates to the semi-static data, the patient data, and provider data.
